(19) United States
(12) Patent Application Publication
(10) Pub. No.: US 2002/0064870 A1
(43) Pub. Date: May 30, 2002

(54) RECOMBINANT ADENOVIRUSES AND USE THEREOF IN GENE THERAPY FOR TREATING EYE DISEASES

(76) Inventors: PASCALE BRIAND, PARIS (FR); MICHEL PERRICAUDET, ECROSNES (FR)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/087,156

(22) Filed: May 28, 1998

Related U.S. Application Data
(63) Continuation of application No. 08/513,998, filed on Oct. 27, 1995.
Application No. 08/513,998 is a 371 of international application No. PCT/FR94/00220, filed on Feb. 28, 1994.

(30) Foreign Application Priority Data
Mar. 3, 1993 (FR) FR93-02438

(51) Int. Cl. C12N 15/63; C12N 15/12

(52) U.S. Cl. 435/320.1; 536/23.5

(57) ABSTRACT

The present invention concerns utilization of defective recombinant viruses containing an inserted gene for preparation of a pharmaceutical composition for treatment of ocular pathologies.

The present invention relates to new recombinant viruses, to their preparation and to their use in gene therapy for the transfer of genes to the eye and their expression therein. It also relates to pharmaceutical compositions comprising the said recombinant viruses. More especially, the present invention relates to defective recombinant viruses and to their use for the treatment of ocular pathologies.

The treatment of ocular pathologies, and in particular of hereditary diseases, constitutes a problem which has not been solved at the present time. Among these pathologies, there may be mentioned, for example, retinitis pigmentosa, which results from adverse genetic modifications and for which no treatment is currently available. Moreover, no suitable treatment is at present available either for nonhereditary pathologies such as post-inflammatory complaints (retinal degeneration, and the like). In particular, while an effort is made to act preventively, in particular using corticoids, no satisfactory means for treating these complaints is currently available.

It is hence important to be able to have tools available permitting a specific, effective and localized treatment of ocular pathologies. The present invention provides an advantageous approach to this problem, by demonstrating the possibility of treating ocular pathologies by gene therapy.

Gene therapy consists in correcting a deficiency or an abnormality (mutation, aberrant expression, and the like) by introduction of genetic information into the affected cell or organ. This genetic information may be introduced either in vitro into a cell extracted from the organ, the modified cell then being reintroduced into the body, or directly in vivo into the appropriate tissue. In this second case, different techniques exist, including various transfection techniques involving complexes of DNA and DEAE-dextran (Pagano et al., J.Virol. 1 (1967) 891), of DNA and nuclear proteins (Kaneda et al., Science 243 (1989) 375), of DNA and lipids (Felgner et al., PNAS 84 (1987) 7413), the use of liposomes (Fraley et al., J. Biol. Chem. 255 (1980) 10431), and the like. More recently, the use of viruses as vectors for the transfer of genes has appeared as a promising alternative to these physical transfection techniques. In this connection, different viruses have been tested for their capacity to infect certain cell populations. These comprise, in particular, retroviruses (RSV, HMS, MMS, and the like), the ESV virus, adeno-associated viruses and adenoviruses.

However, hitherto, none of these vectors has been used or described as being usable for the transfer of genes to the eye. The present invention constitutes the first demonstration that it is possible to treat ocular pathologies by gene therapy.

A first subject of the invention lies in the use of a defective recombinant virus containing an inserted gene for the preparation of a pharmaceutical composition intended for the treatment of ocular pathologies.

More especially, defective recombinant viruses derived from viruses capable of infecting and of expressing an inserted gene in the cells of the eye, without giving rise to cytopathological phenomena or pathogenic effects, are used according to the present invention. Viruses capable of being used in the invention are, for example, adenoviruses, adeno-associated viruses or alternatively the ESV virus.

The present invention is based more especially on the demonstration that adenovirus type viruses are capable of transferring desired genes to the eye and of expressing them therein. The examples presented later show that adenoviruses are capable, depending on the mode of administration, of transferring genes to the corneal endothelium, to photoreceptor cells, to cells of the optic nerve, to bipolar cells, and the like, effectively, for a considerable period and without a cytopathological effect. Moreover, in view of the relative ease of access to the different compartments of the eye by microsurgery (microinjection), as well as of the existence of natural barriers in this organ (Descemet's membrane, Bruch's membrane, lens, and the like), the present invention advantageously enables a very targeted transfer of genes to be performed, in accordance with the pathology to be treated. The results presented also show that the expression of a desired gene is stable over a long period (no loss of activity at 50 days).

In a preferred embodiment, the invention lies in the use of a defective recombinant adenovirus containing an inserted gene for the preparation of a pharmaceutical composition intended for the treatment of ocular pathologies.

The term “defective virus or adenovirus” denotes a virus incapable of replicating autonomously in the target cell. Generally, the genome of the defective viruses used in the context of the present invention hence lacks at least the sequences needed for replication of the said virus in the infected cell. These regions may be either removed (wholly or partially), or rendered non-functional, or replaced by other sequences, and in particular by the inserted gene. Preferably, the defective virus nevertheless retains the sequences of its genome which are needed for encapsidation of the viral particles.

Regarding, more especially, adenoviruses, the latter exist in the form of different serotypes, whose structure and properties vary somewhat. Nevertheless, these viruses are not pathogenic for man, and in particular non-immunosuppressed subjects. Among these serotypes, it is preferable, in the context of the present invention, to use adenovirus type 2 or 5 (Ad 2 or Ad 5). In the case of adenoviruses Ad 5, the sequences needed for replication are the E1A and E1B regions.

Defective recombinant viruses derived from retroviruses, from adeno-associated viruses or from the HSV virus (herpes simplex virus) have already been described in the literature [Roemer and Friedmann, Eur. J. Biochem. 208 (1992) 211; Dobson et al., Neuron 5 (1990) 353; Chiocca et al., New Biol. 2 (1990) 739; Miyanohara et al., New Biol. 4 (1992) 238; WO91/18088].

For the purposes of the present invention, the term “inserted gene” denotes any DNA sequence introduced into the recombinant virus, whose expression in the target cell is sought.

It can be, in particular, one (or more) structural gene(s) coding for a (some) protein(s) or for a portion of a (some) protein(s). The protein or protein portion thus encoded can be a protein which is homologous with respect to the target cell (that is to say a protein which is normally expressed in the target cell when the latter does not exhibit any pathology), or a protein which is heterologous with respect to the said cell. In the former case, expression of the protein makes it possible, for example, to remedy an insufficient expression in the cell or the expression of a protein which is inactive or poorly active as a result of a modification, or alternatively to overexpress the said protein. In the second case, the protein expressed can, for example, supplement or supply an activity which is deficient in the cell, enabling it to combat a pathology.

Among inserted genes for the purposes of the present invention, there may be mentioned, more especially:

genes involved in ocular genetic pathologies,

genes coding for growth factors, cytokines or neurotrophins: the protective or curative role of the expression product of these genes in different ocular pathologies has been demonstrated, and in particular on the deterioration of photoreceptor cells under the effect of light (Lavail et al., PNAS 89 (1992) 11249),

genes for regulatory factors (transcription factors, translation factors),

genes coding for enzymes,

genes coding for proteins having anticancer properties, such as interferons, tumour necrosis factors, and the like, or alternatively,

genes coding for antigens permitting a local vaccination (protection) against an eye infection.

As specific, but non-limiting, examples, there may be mentioned:

the ornithine aminotransferase gene involved in gyrate atrophy (Akaki et al., J. Biol. Chem. 267 (18) (1992) 12950),

the rhodopsin gene involved in a form of retinitis pigmentosa (Dryja et al., Nature 343 (1990) 364),

the RDS peripherin gene involved in a form of retinitis pigmentosa (Farrar et al., Nature 354 (1991) 478),

the tyrosinase gene involved in type B1 oculocutaneous albinism (Giebel et al., Am. J.Hum. Genet. 48 (1991) 1159),

the mitochondrial NDI gene involved in Leber's disease (Howell et al., Am. J. Hum. Genet. 48 (1991) 935),

the gene for the β subunit of cGMP phosphodiesterase, which enables retinal degeneration to be slowed down (Lem et al., PNAS 89 (1992) 4422),

the rab geranylgeranyl transferase gene, the deficiency of which appears to be associated with a retinal degeneration in choroidermia (Seabra et al., Science 259 (1993) 377),

the basic fibroblast growth factor (bFGF) gene, capable of retarding the degeneration of the photoreceptor cells which is observed in some hereditary retinal dystrophies (Faktorovich et al., Nature 347 (1990) 83),

the interleukin-8 gene, which enables a neovascularization to be induced in the cornea (Strieter et al., Am. J. Pathol. 141 (6) (1992) 1279).

The term “inserted gene” also denotes antisense sequences, whose expression in the target cell enables the expression of genes or the transcription of cellular mRNAs to be controlled. Such sequences can, for example, be transcribed in the target cell into RNAs complementary to cellular mRNAs and can thus block their translation into protein.

Generally, the inserted gene also comprises sequences permitting its expression in the infected cell. The sequences in question can be ones which are naturally responsible for expression of the said gene when these sequences are capable of functioning in the infected cell. They can also be sequences of different origin (responsible for the expression of other proteins, or even synthetic sequences). In particular, they can be sequences originating from the genome of the cell which it is desired to infect, or from the genome of the virus used. In the case of adenoviruses, there may be mentioned, for example, the promoters of the E1A, MLP genes, and the like. In addition, these expression sequences may be modified by the addition of activation, regulatory, and the like, sequences. Moreover, when the inserted gene does not contain expression sequences, it may be inserted into the genome of the defective virus downstream of such a sequence.

In what follows, the construction and use of defective recombinant adenoviruses are described in greater detail. It is nevertheless understood that this description may be applied by a person skilled in the art to other viruses capable of being used in the context of the present invention, as mentioned above.

Defective recombinant adenoviruses may be prepared by homologous recombination between an adenovirus and a plasmid carrying, inter alia, the gene which it is desired to insert. Homologous recombination takes place after cotransfection of the said adenovirus and said plasmid into a suitable cell line. The cell line used should preferably (i) be transformable by the said elements, and (ii) contain the sequences capable of complementing the portion of the genome of the defective adenovirus, preferably in integrated form in order to avoid the risks of recombination. As an example of a line, there may be mentioned the human embryonic kidney line 293 (Graham et al., J. Gen. Virol. 36 (1977) 59), which contains, in particular, integrated in its genome, the left-hand portion of the genome of an adenovirus Ad 5 (12%).

Thereafter, the vectors which have multiplied are recovered and purified according to standard techniques of molecular biology.

The present invention also relates to a pharmaceutical composition comprising a sufficient amount of defective recombinant virus as defined above, in a form suitable for ocular administration.

In particular, the defective recombinant virus may be in the form of an injection, eye lotion, ophthalmic ointment, and the like. The pharmaceutically acceptable vehicles for such formulations suitable for ocular administration are, in particular, saline solutions (monosodium or disodium phosphate, sodium, potassium calcium or magnesium croride, and the like, or mixtures of such salts), soft paraffin, liquid paraffin, and the like.

In the case of eye lotions or ophthalmic ointments, it is understood that the therapeutic applications may be more limited on account of a weaker diffusion of the defective recombinant virus.

In their use for the treatment of ocular pathologies, the defective recombinant viruses according to the invention may be administered according to different modes, and in particular by subretinal injection preceded, where appropriate, by a vitrectomy, or by intravitreous injection, the injections being single or multiple (see FIG. 1). Subretinal injection may be carried out selectively in different compartments of the eye, and, in particular, injection may be carried out in the vitreous, in the anterior chamber or in the retrobulbar space. The results presented in the present application show that these different modes of injection enable the different tissues of the eye, and in particular the corneal endothelium, the photoreceptor cells, the bipolar cells, the ganglion cells or alternatively the cells of the oculomotor muscles, to be infected in a targeted manner.

The doses of virus used for the injection may be adapted in accordance with different parameters, and in particular in accordance with the mode of administration used, the pathology in question, the gene to be expressed or alternatively the period of treatment required. Generally speaking, the recombinant adenoviruses according to the invention are formulated and administered in the form of doses of between 10⁴ and 10¹⁴ pfu/ml, and preferably 10⁶ to 10¹⁰ pfu/ml. The term pfu (plaque forming unit) corresponds to the infectious power of a solution of virus, and is determined by infection of a suitable cell culture and measurement, generally after 48 hours, of the number of plaques of infected cells. The techniques of determination of the pfu titre of a viral solution are well documented in the literature.

In view of the stability of expression of the inserted gene in the target cell, the present invention should make it possible to treat the majority of ocular pathologies with few injections.

The present invention thus affords a very effective means for the treatment of ocular pathologies, and in particular those whose mechanisms have been elucidated at molecular level. In particular, the involvement of genes has been demonstrated in gyrate atrophy, in Norrie's disease (Hum. Mol. Genet. 1 (7) (1992) 461), in retinal degeneration (Bowes et al., PNAS 86 (1989) 9722) in Leber's disease, in choroidermia (Cremers et al., Nature 347 (1990) 674), in degeneration of photoreceptor cells, in retinitis pigmentosa, in albinism, in Kearns-Sayre syndrome (Shoffner et al., PNAS 86 (1989) 7952), and the like. The present invention is also for the treatment of acquired adverse modification in the cornea resulting from inflammatory disorders, post-inflammatory retinal complaints, and the like.

The present invention also makes possible therapy with proteins or peptides, the use of which via the traditional administration routes is very hypothetical on account of their great sensitivity to the mechanisms of degradation and elimination from the body, and problems associated with penetration into the cells. The use of viruses according to the invention permits the direct expression, within the population of targeted cells, of the desired protein or polypeptide, which is hence no longer accessible to the mechanisms mentioned above.

The collective results presented in the present application demonstrate, more especially, that recombinant adenoviruses, which are defective for replication, constitute especially advantageous vectors for the transfer of genes in vivo to ocular cells. The experiments carried out show the possibility of a stable long-term expression of genes in these cells. In particular, a stable expression is observed 50 days after injection. Furthermore, the broad spectrum of expression in the different ocular cells also constitutes an especially advantageous result, inasmuch as practically all disorders of the retina (in particular retinitis pigmentosa) affect a large area of the retina.

In addition, this treatment can relate both to man and to any animal such as sheep, cattle, domestic animals (dogs, cats, and the like), horses, fish, and the like.

The present invention is described more completely by means of the examples which follow, which are to be considered as illustrative and non-limiting.

### LEGEND TO THE FIGURES

FIG. 1: Diagrammatic representation of the eye. C=cornea; AC=anterior chamber; L=lens; V=vitreous; I=iris; ON=optic nerve; R=retrobulbar space.

Construction of a Defective Recombinant Adenovirus (Ad.RSVβGal):

The general procedure enabling recombinant adenoviruses to be prepared has been described in the general part of the description.

The adenovirus Ad.RSVβGal is a defective recombinant adenovirus (from which the E1 and E3 regions have been deleted) obtained by homologous recombination in vivo between the mutant adenovirus Ad-d1324 (Thimmappaya et al., Cell 31 (1982) 543) and plasmid pAd.RSVβGal (Akli et al., 1993).

Plasmid pAd.RSVβGal contains, in the 5′→3′ orientation,

the PvuII fragment corresponding to the left-hand end of the adenovirus Ad 5, comprising: the ITR sequence, the origin of replication, the encapsidation signals and the E1A amplifier;

the gene coding for β-galactosidase under the control of the RSV (Rous sarcoma virus) promoter;

a second fragment of the genome of the adenovirus Ad 5, which permits homologous recombination between plasmid pAd.RSVβGal and the adenovirus d1324.

After linearization with the enzyme ClaI, plasmid pAd.RSVβGal and the adenovirus d1324 are cotransfected into the line 293 in the presence of calcium phosphate to permit homologous recombination. The recombinant adenoviruses thus generated are selected by purification on plates. After isolation, the DNA of the recombinant adenovirus is amplified in the cell line 293, thereby leading to a culture supernatant containing the unpurified recombinant defective adenovirus having a titre of approximately 10¹⁰ pfu/ml.

The viral particles are generally purified by centrifugation on a caesium chloride gradient according to known techniques (see, in particular, Graham et al., Virology 52 (1973) 456). The adenovirus Ad.RSVβGal is stored at −80° C. in 20% glycerol. Before injection, the adenovirus suspension is diluted to one third in phosphate buffer PBS.

Injection in Vivo

Protocol

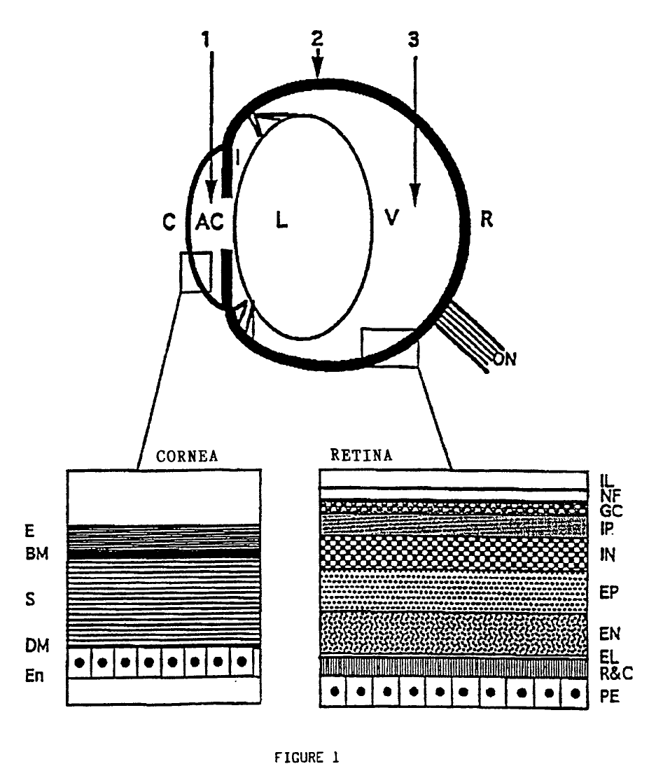

3- to 7-week-old C57B1/6 mice were anaesthetized with Avertin. 10⁷ to 10⁸ pfu of recombinant adenovirus Ad.RSVβGal were then injected into each eye, either in the anterior chamber, or in the vitreous, or in the retrobulbar space (see FIG. 1). The animals were sacrificed 7 to 50 days after injection by cervical dislocation, and the eyes were recovered and fixed in liquid nitrogen. Sagittal and coronal sections (10-15 μm) are prepared on a cryostat, then stained in the presence of X-gal to disclose β-galactosidase activity, which may be visualized by the appearance of a blue stain in the nucleus of the infected cells, and counterstained with haematoxylin and eosin.

Injection in the Anterior Chamber

After injection of 10⁸ pfu of adenovirus Ad.RSVβGal in the space of the anterior chamber, only the cells of the endothelial layer express β-galactosidase activity. On the other hand, the epithelial or stromal cells do not exhibit any staining following such an injection. Furthermore, the labelled (infected) cells are distributed evenly in the endothelial layer, irrespective of the time of administration. This result shows that the present invention enables a gene to be transferred to the endothelial cells of the eye and expressed therein.

Intravitreous Injections

Intravitreous injections were also carried out, with the object of infecting different cell types of the retina. In contrast to the uniform distribution in the endothelial cells after injection in the anterior chamber space, the distribution of positive (infected) cells after intravitreous injection is limited to the half-retina corresponding to the point of injection. The large size of the lens and the viscosity characteristics of the vitreous humour might explain this confined expression. However, when temporal and nasal injections are performed simultaneously, the cells of both half-retinas are infected. Hence these results show that it is possible to transfer a gene to the retina and express it therein. They also show that, depending on the pathology to be treated and, in particular, depending on its distribution on the retina, it is possible to target the transfer on one half-retina only.

Three nuclear layers, corresponding to the ganglion, bipolar and photoreceptor cells, also exhibit an intense staining at three weeks (age at which development of the retina is complete), as well as in adult mice. Despite the presence of the signal permitting the nuclear localization of the LacZ protein, the labelling (and hence infection) of some cells at the injection site is so intense that the staining diffuses into the cytoplasm. For this reason, the layer of nerve fibre corresponding to the axons of the labelled nuclei (which converge to form the optic nerve) is labelled homogeneously.

A careful analysis of the different layers of retinal cells does not reveal any significant decrease in their thickness. Furthermore, the head of the optic is nerve is not adversely affected, even at high doses of adenovirus (10⁷ pfu).

Injection in the Retrobulbar Space

To evaluate the possibility of a diffusion of the virus through the sclera, mice were injected in the retrobulbar space. In contrast to the retinal staining, approximately 100% of the fibres of the 4 oculomotor muscles were infected and express β-galactosidase activity.

These collective results clearly demonstrate that recombinant adenoviruses which are defective for replication constitute especially advantageous vectors for the transfer of genes in vivo to ocular cells.

1. Use of a defective recombinant adenovirus containing an inserted gene for the preparation of a pharmaceutical composition intended for the treatment of ocular pathologies.

2. Use according to claim 1, characterized in that the defective recombinant adenovirus lacks the regions of its genome which are needed for its replication in the infected cell.

3. Use according to claims 1 or 2, characterized in that the defective recombinant adenovirus is a type Ad 2 adenovirus.

4. Use according to claim 1 or 2, characterized in that the defective recombinant adenovirus is a type Ad 5 adenovirus.

5. Use according to one of claims 1 to 4, characterized in that the inserted gene comprises sequences permitting its expression in the infected cell.

6. Use according to one of claims 1 to 5, characterized in that the inserted gene codes for a protein or a protein fragment.

7. Use according to one of claims 1 to 5, characterized in that the inserted gene is an antisense sequence.

8. Use according to claim 1 for the preparation of a pharmaceutical composition intended for the treatment of hereditary pathologies such as retinitis pigmentosa.

9. Pharmaceutical composition comprising a sufficient amount of a defective recombinant adenovirus according to claim 1, in a form suitable for ocular administration.

10. Pharmaceutical composition according to claim 9, characterized in that it comprises a sufficient amount of defective recombinant adenovirus in an injectable form suitable for ocular administration.

11. Pharmaceutical composition according to claim 9, characterized in that it comprises a sufficient amount of defective recombinant adenovirus in the form of an eye lotion or ophthalmic ointment suitable for ocular administration.

12. Pharmaceutical composition according to one of claims 9 to 11, characterized in that the defective recombinant adenovirus is a defective recombinant type Ad 2 or Ad 5 adenovirus.

13. Pharmaceutical composition according to claim 12, characterized in that it comprises between 10⁴ and 10¹⁴ pfu/ml, and preferably 10⁶ to 10¹⁰ pfu/ml, of defective recombinant adenovirus.
